(19) United States
(12) Patent Application Publication
(10) Pub. No.: US 2026/0366987 A1
(43) Pub. Date: Nov. 19, 2026

(54) SYSTEMS AND METHODS FOR DETERMINING STABILITY OF DRUG FORMULATIONS

(71) Applicant: Unchained Labs, LLC, Pleasanton, CA (US)

(72) Inventors: Edward Joseph MILOVIC, Pleasanton, CA (US); Kai Wah SZETO, Berkeley, CA (US); Lee JEFFRIES, Caxton (GB); Philip James HOMEWOOD, Enfield (GB); Kenneth John CUNNINGHAM, High Wycombe (GB); David Wesley WYRICK, Livermore, CA (US)

(21) Appl. No.: 19/439,829

(22) Filed: Jan. 5, 2026

Related U.S. Application Data
(60) Provisional application No. 63/742,219, filed on Jan. 6, 2025.

(51) Int. Cl. G01N 21/03; B01L 3/00; G01N 21/47; G01N 21/64

(52) U.S. Cl. CPC G01N 21/0332; B01L 3/502; B01L 2200/026; B01L 2200/028; B01L 2200/0684; B01L 2300/18; B01L 2400/0478; G01N 21/47; G01N 21/64

(57) ABSTRACT

Described herein are systems and methods that combine at least three different detection modalities to characterize the stability of drug formulations, and sample plates for use with the systems and methods. The systems may include a clamp configured to apply a force to the sample plates that trap and pressurize air above an inlet and/or outlet of sample holders included in the sample plates. Measurements of various parameters of the samples may be obtained in tandem or simultaneously with a single instrument, which reduces sample volume requirements.

### CROSS-REFERENCE TO RELATED APPLICATION

This application claims priority to U.S. Provisional Application No. 63/742,219, filed on Jan. 6, 2025, which is hereby incorporated by reference in its entirety.

### FIELD

Described herein are systems and methods for characterizing the stability of drug formulations. The systems and methods may employ a sample plate configured to allow different types of optical measurements to be made sequentially or simultaneously using a single instrument. The sample plate may further be configured to allow high-throughput, automated determination of drug stability including illumination and/or heating of a plurality of small volume drug samples. The information acquired by the systems may include thermodynamic, kinetic, and/or light scattering data from the samples.

### BACKGROUND

Assessing drug stability is a crucial step in the drug development process. This is true for all drug constructs, whether they are biologic, viral vector, or non-viral vector-based drugs. Stability testing may help identify viable drug candidates as well as determine formulation components and storage conditions that maintain stability of a drug over its shelf life. In some instances, stability may be assessed by applying a stress (e.g., heat) to a drug and monitoring its structure and quality. Given that structure is directly linked to activity, as the drug unfolds or dissociates, it will generally become less active. Aggregation and precipitation are also mechanisms of inactivation for drugs that are of concern.

A range of analytical techniques employing optical methods are available to provide information relating to unfolding, aggregation, and precipitation for substances of interest. Typically, the samples including one or more substances of interest are illuminated with light and, depending on the technique employed, the resulting emitted, scattered, or transmitted light is collected, spectrally analyzed as appropriate, and then detected using some form of optical detector. Optical analytical techniques in common use include fluorescence spectroscopy, optical absorption spectroscopy, infra-red absorption spectroscopy, light scattering, and Raman spectroscopy. Each form of optical analytical technique may provide different information about a substance of interest, and it is therefore often advantageous to perform multiple different forms of optical analysis on a given sample in order to provide a deeper understanding of the properties of the sample.

Conventional sample containers that allow the performance of individual optical measurements from single liquid samples are generally described as “cuvettes”. Although cuvettes are available for a wide range of sample volumes, these sample volumes are generally large, e.g., between about 50 μl to about 100 μl. Widely used multi-container arrays are 96 well, 384 well, and 1536 well (microtiter) plates conforming to standards published by the Society for Biomolecular Screening. These may be either viewed from the top or in some cases through the bottom of the well. These sample plates do not, however, allow illumination and collection of light at a wide range of angles, and thus the optimal configuration for many of the optical analytical techniques cannot be achieved. In some cases, a large sample volume must be used to compensate for the sub-optimal optical configuration.

In applications such as protein stability measurements, samples are available in only very small quantities and are not reusable between tests, which can prevent the separate application of multiple optical measurements. It would therefore be useful if multiple types of optical measurements could be performed sequentially or simultaneously on small sample volumes. Additionally, given the demand for high-throughput determinations of drug and other substance stability, it would be beneficial to have new systems, sample plates, and methods for use with automated processes to improve workflow.

### SUMMARY

Described herein are systems and methods that combine multiple (e.g., at least two, at least three) different detection modalities to characterize the stability of drug formulations, and sample plates for use with the systems and methods. The systems may include a clamp configured to apply a force to the sample plates and trap and pressurize air above an inlet and/or outlet of sample holders included in the sample plates. Measurements of various parameters of the samples may be obtained sequentially or simultaneously with a single instrument, which may reduce sample volume requirements. The systems may also be partially or fully automated. For example, the systems may be configured to work with robotic arms and other mechanisms for robotic loading of samples into sample plates.

The systems for analyzing the stability of samples may generally include an optical module and a sample plate including one or more substrates. The one or more substrates may include a plurality of sample holders (e.g., cuvettes) and an inlet fluidly coupled to each of the plurality of sample holders. Additionally, the systems may include a clamp having an actuated (e.g., clamped) state in which it is configured to trap and pressurize air above the inlet.

Multiple modules and sub-modules configured to measure various sample parameters may be included in the systems such that a single instrument may run several experiments sequentially or simultaneously. Put another way, multiple modules and sub-modules may be contained within a single housing of an instrument that may run several experiments one after the other or at the same time. For example, an optical module within the instrument housing may comprise a first sub-module (a first apparatus) configured to measure fluorescence of a sample within at least a portion of the plurality of sample holders. The first sub-module may also measure static light scattering (SLS) of the sample within at least a portion of the plurality of sample holders, or the SLS may be measured using a separate, second sub-module (a second apparatus). A third sub-module (a third apparatus) configured to measure dynamic light scattering (DLS) of a sample within at least a portion of the plurality of sample holders may further be included. In some instances, the systems may also a temperature control unit for heating the samples. The systems may further include a display configured to visually present data obtained from the optical module and/or one or more processors coupled to the optical module.

Sample plates that may be used with the systems may generally be configured to allow multiple experiments to be run on a single low volume sample, e.g., a volume of about 9.0 μl, with a single instrument. The sample plate may include a frame having length and a width coupled to the one or more substrates. For example, the frame may have a length between about 120 mm and about 130 mm, including all values and sub-ranges therein, and a width between about 80 mm and about 90 mm, including all values and sub-ranges therein. Various types of polymers may be used to make the frame. In one variation, the frame may be made from Polycarbonate Acrylonitrile Butadiene Styrene (PC-ABS).

The sample plates may include any number of sample holders (e.g., cuvettes). For example, the sample plate may be sized and shaped to include 96 sample holders. When a sample of interest is to be tested, each sample holder is loaded with a sample containing a target drug, protein, viral vector, or a combination thereof. The plurality of sample holders may be variously shaped. In some variations, the plurality of sample holders may have a diamond, rounded diamond, marquis, ovular, circular, rectangular, or square shape.

Some variations of the sample plate may include six substrates attached to the frame.

Attachment may be accomplished via one or more pins on the frame that fit into one or more notches in each of the substrates. When six substrates are employed, the substrates may have a length between about 70 mm to about 80 mm, including all values and sub-ranges therein, and a width between about 10 mm to about 15 mm, including all values and sub-ranges therein.

The one or more substrates may comprise a plurality of substrate layers. Any number of substrate layers may be included. Additionally, any material that does not interfere with fluorescence and optical readings, and which may form an atomically flat surface to optimize the transfer of heat to the samples may be used to make the substrate layers. In some variations, each of the plurality of substrate layers may be made of a quartz material. For example, JGS2 quartz or JGS1 quartz may be used. In other variations, polymer materials capable of effectively transmitting UV light, e.g., UV light between 280 nm and 660 nm and at a transmission rate of about 85% to about 95% may be used. Exemplary polymers that transmit UV light include without limitation, cyclic olefin copolymer (COC) (e.g., a Topas® COC polymer), ultraviolet transmission (UVT) acrylic (e.g., poly(methyl methacrylate) (PMMA)), and ultraviolet transmission (UVT) polycarbonate.

Each of the plurality of substrate layers may have a thickness between about 0.5 mm to about 1.0 mm, including all values and sub-ranges therein. For example, the thickness of each substrate layer may be about 0.5 mm, about 0.6 mm, about 0.7 mm, about 0.8 mm, about 0.9 mm, or about 1.0 mm. In one variation, each of the plurality of substrate layers may have a thickness of about 0.7 mm.

When the substrate includes three layers, it may comprise a top layer, a base layer, and a middle layer between the top and base layers. In this configuration, the top layer may include the inlet for loading the sample on a first side of each sample holder (e.g., cuvette) and a vent outlet on a second, opposite side of each sample holder, and the base layer may comprise one or more notches at an edge thereof configured to mate with one or more pins on the sample plate. The middle layer may include the plurality of sample holders (e.g., cuvettes), and an inlet channel and a vent channel extending from each of the plurality of sample holders. The inlet channel may fluidly connect the inlet to each of the plurality of sample holders, and the vent channel may fluidly connect a vent outlet to each of the plurality of sample holders. In some variations, each of the plurality of sample holders (e.g., cuvettes) and its corresponding inlet channel and vent channel may be sized and/or shaped to collectively hold a small sample volume, e.g., a sample volume of about 9.0 μl.

When the aqueous samples being tested require heating, e.g., heating to about 95 degrees Celsius, the systems and/or sample plates generally include mechanisms to minimize evaporation and bubble formation. For example, the systems may employ a plate seal configured to cover at least a portion of the one or more substrates and adhesively attach to a frame of the sample plate. The plate seal may comprise one or more elongate openings disposed over the plurality of sample holders when the plate seal is adhesively attached to the frame of the sample plate. In addition to the plate seal, the systems may include a clamp having an actuated state and a rest state. The clamp may comprise a plurality of pneumatic pistons that allow the clamp in its actuated state to apply a force between about 300 kPa (3 bar) to about 500 kPa (5 bar), including all values and sub-ranges therein, to the sample plate. In the actuated state, the clamp may trap and pressurize air above an inlet fluidly coupled to each of the plurality of sample holders. In some variations, the clamp may also trap and pressurize air above a vent outlet fluidly coupled to each of the plurality of sample holders.

In one variation, the sample plate may include a frame and a plurality of substrates coupled to the frame, where each of the plurality of substrates includes a top layer, a base layer, and a middle layer between the top and base layers, and where the middle layer a plurality of sample holders (e.g., cuvettes), and the top layer includes an inlet and a vent outlet fluidly connected to each of the plurality of sample holders. In this variation, each of the plurality of sample holders may be fluidly connected to the inlet by an inlet channel disposed within the middle layer, and each of the plurality of sample holders may be fluidly connected to the vent outlet by a vent channel disposed within the middle layer. The plurality of sample holders may have a diamond, rounded diamond, or marquis shape. Each of the plurality of sample holders may be configured to hold a sample volume between about 8.0 μl to about 9.0 μl.

In some variations, the system may include a sample holder configured to receive a sample, the sample including a protein component. The optical module of the system may include a first apparatus (e.g., sub-module) configured to detect a thermodynamic state of the first portion of the sample. The system may also include a second apparatus (e.g., sub-module) configured to modify the temperature of the sample, and to detect, in response to the modifying the temperature, a second measure from the second portion of the sample. The second measure may be indicative of a rate of denaturation of the protein of the sample.

The system may include one or more processors communicably coupled to the optical module and/or sub-modules. The one or more processors may be configured to receive the information measured by the optical module and/or sub-modules, and based on that information, compute an indication of the stability of the protein of the sample. Instead of proteins, the one or more processors may also base its analysis of stability on information obtained for other substances such as viral vectors, biologics, or chemicals.

Methods for analyzing the stability of samples are also described herein. The methods may generally include loading a sample into a a plurality of sample holders of a sample plate, where the sample plate includes a frame and one or more substrates coupled to the frame, and where the one or more substrates includes a top layer, a base layer, and a middle layer between the top and base layers. A clamp may then be actuated to apply a force to the sample plate and form a seal that pressurizing air trapped above an inlet of the each of the plurality of sample holders. The force applied by the clamp may be between about 300 kPa (3 bar) to about 500 kPa (5 bar), including all values and sub-ranges therein. In some instances, the method may include pressurizing air trapped above a vent outlet of each of each of the plurality of sample holders. The loading of the samples may be partially automated or fully automated. The samples of interest may include any suitable target substance to be assessed, including drug, proteins, viral vectors, biologics, chemicals, or combinations thereof.

Some variations of the method may further include adhesively attaching a plate seal on the sample plate to cover at least a portion of the one or more substrates, e.g., when the method includes heating of the samples. Additionally, the methods may include one or more steps of detecting fluorescence of the sample, detecting static light scattering (SLS), and detecting dynamic light scattering (DLS) of the sample. The data obtained from these various detection methods may be displayed on a screen as, e.g., numerical data, lists, charts, graphs, or other visual (e.g., color-coded) representations of the information.

In other variations, the method may include receiving a sample, the sample including a drug (e.g., a protein) component. The method may include detecting a thermodynamic state of the sample. In some variations, a denaturing agent may be added to the sample prior to detecting the thermodynamic state. The method may also include modifying the temperature of the sample and detecting, in response to the modifying the temperature, a rate of denaturation of a protein in the sample. The method also includes computing the thermodynamic information and the kinetic information using one or more processors, to provide an indication of stability of the protein component of the sample. As mentioned above, instead of proteins, the one or more processors may also base its analysis of stability on information obtained for other substances such as viral vectors, biologics, or chemicals.

### BRIEF DESCRIPTION OF THE DRAWINGS

FIG. 1 is a schematic block diagram of an exemplary system for assessment of substance stability.

FIG. 2 depicts an exemplary optical module for assessment of substance stability.

FIG. 3A depicts an exemplary sample plate including a frame and a plurality of substrates (chips).

FIG. 3B depicts a wing-shaped hole useful for when an adhesive is employed to attach the substrates shown in FIG. 3A to a frame.

FIG. 4A depicts exemplary top, middle, and base layers of a substrate (chip). Further details of notches, sample holders (e.g., cuvettes), inlets, inlet channels, vent outlets, and vent channels are shown in FIGS. 4B to 4E.

FIGS. 5A to 5D depict exemplary system components configured to control temperature and minimize evaporation of the samples, and minimize bubble formation in the samples.

### DETAILED DESCRIPTION

Described herein are systems and methods that may be used to analyze the stability of various substances, e.g., drug formulations, proteins, or viral vectors. For example, the systems and methods may be used to quantitatively predict the long-term stability (i.e., stability over time) of substances. The systems and methods may generally monitor stability by assessing: 1) increasing intrinsic fluorescence as proteins unfold; 2) aggregation and precipitation by static light scattering (SLS); and 3) size change and aggregation by dynamic light scattering (DLS). The systems and methods may integrate thermodynamic, kinetic, and/or light scattering information with respect to the appearance, growth, and/or accumulation of denatured state aggregates. The intrinsic fluorescence, SLS, and DLS may be assessed sequentially or simultaneously using a collection of modules and sub-modules contained within a single instrument.

### SYSTEM OVER VIEW

The systems for analyzing the stability of a substance may be a benchtop instrument including a wide range of substance (e.g., protein) characterization capabilities. The system may generally combine several types of measurements, e.g., fluorescence, static light scattering (SLS), and dynamic light scattering (DLS), which may allow the system to sequentially or simultaneously assess factors related to substance (e.g., protein) stability and aggregation using a single instrument and/or a single sample, which may reduce sample volume requirements. The systems may be partially or fully automated. For example, the systems may be configured to work with robotic arms and other mechanisms for robotic loading of samples into sample plates, as previously mentioned.

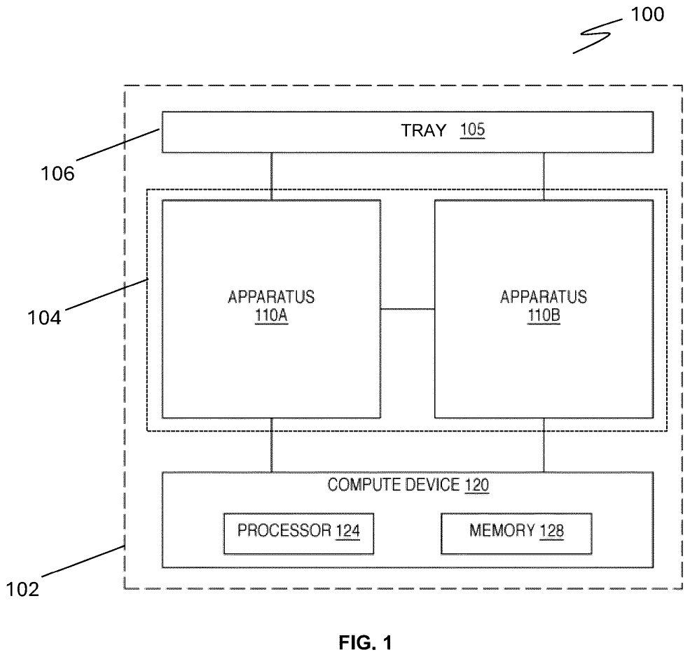

In some variations, the system may be configured as shown in FIG. 1. Referring to FIG. 1, system 100 may include an instrument housing 102 containing a tray 105 for receiving a sample plate including a plurality of sample holders, an optical module 104 comprising a first apparatus (first sub-module) 110A and a second apparatus (second sub-module) 110B, and a compute device 120. A sample plate is typically placed on the tray 105 and then moved into the instrument housing 102 after the samples have been loaded onto the sample plate. Although shown as being disposed within the instrument housing 102, some components of the compute device 120, e.g., one or more processors, may be located outside the instrument housing 102.

The compute device 120 may be, for example, a server, a computer, a router, a data storage device, a tablet, and/or a mobile device. The compute device 120 may include computer software (stored in and/or executed at hardware) such as a web application, a database application, a cache server application, a queue server application, an operating system, a file system, and/or the like; computer hardware such as a network appliance, a storage device (e.g., disk drive, memory module), a processing device (e.g., computer central processing unit (CPU)), a computer graphic processing unit (GPU), and/or a networking device (e.g., network interface card); and/or combinations of computer software and hardware. The compute device 120 may be operatively coupled to the optical module 104, first and/or second apparatuses (first and/or second sub-modules) 110A and 110B, respectively, and/or other devices.

As shown in FIG. 1, the compute device 120 may include a memory 128 and a processor 124, and may also include other component(s) (not shown in FIG. 1), such as, for example, a display, an interface to permit a user/operator to interact with the compute device 120 and/or the system 100. The memory 128 may be, for example, a Random-Access Memory (RAM) (e.g., a dynamic RAM, a static RAM), a flash memory, a removable memory, and/or so forth. In some instances, instructions associated with performing the operations described herein (e.g., computing long-term stability) may be stored within the memory 128 and executed at the processor 124.

In some variations, each module/component in the processor 124 may be any combination of a hardware-based module/component (e.g., a field-programmable gate array (FPGA), an application specific integrated circuit (ASIC), a digital signal processor (DSP)), a software-based module/component (e.g., a module of computer code stored in the memory 128 and/or executed at the processor 124), and/or a combination of hardware-and software-based modules/components. Each module/component in the processor 124 may be capable of performing one or more functions/operations that analyze the samples, e.g., to obtain data relating to fluorescence, static light scattering (SLS), and/or dynamic light scattering (DLS). In some instances, the modules/components included and executed in the processor 124 may be, for example, a process, an application, a virtual machine, and/or some other hardware or software module/component (stored in memory and/or executing in hardware). The processor 124 may be any suitable processor configured to run and/or execute such modules/components. One or more processors configured as described for processor 124 herein may be included in the systems.

In other instances, the processor 124 may be configured for simultaneously performing multiple computing tasks for multiple systems and/or processes. In these instances, the compute device 120 may include a communication interface (e.g., a data port, a wireless transceiver and an antenna) to enable data transmission between the compute device 120 and other devices and/or the apparatuses (sub-modules) 110A, 110B. In some instances, the compute device 120 may include or be coupled to a display device (e.g., a printer, a monitor, a speaker, etc.), such that an output of the compute device may be presented to a user via the display device.

In some variations, the compute device 120 and/or the system 100 may be operatively coupled to other devices via, for example, a network. The network may be any type of network that may operatively connect and enable electronic transmission therebetween. The network may be, for example, a wired network (an Ethernet, local area network (LAN), etc.), a wireless network (e.g., a wireless local area network (WLAN), a Wi-Fi network, etc.), or a combination of wired and wireless networks (e.g., the Internet, etc.).

As described above, the tray 105 is typically configured to receive a sample plate. The tray 105 may include any suitable receiving interface for receiving the sample plate. In some instances, instead of tray 105, the receiving interface may be a cartridge holder or a test tube holder. When sample plates are used, the plates may be configured to snap into place on the tray 105.

In some variations, the first apparatus (first sub-module) 110A may be configured to supply a denaturing agent to the samples. The first apparatus (first sub-module) 110A may be fluidly coupled to a source of denaturing agent such as urea, or to one or more sources of suitable acids such as acetic acid, bases such as sodium bicarbonate, solvents such as ethanol, cross-linking agents such as formaldehyde, chaotropic agents such as urea, disulfide bond reducers such as 2-mercaptoethanol, and/or the like. Accordingly, the first apparatus (first sub-module) 110A may include fluid handling components, such as a pump and tubing, to deliver the denaturing agent to the samples.

Additionally or alternatively, the first apparatus (first sub-module) 110A may be configured to excite the sample with an excitation light (e.g., from a suitable light source such as LEDs, a laser, and/or the like), and detect excitation and fluorescence at wavelengths corresponding to intrinsically fluorescent amino acids such as tryptophan, tyrosine, and/or phenylalanine. For example, fluorescence intensity, and/or a derivative thereof (e.g., scaled fluorescence intensity, fluorescence lifetime) may be measured.

The first apparatus (first sub-module) 110A may be configured to maintain the sample at a substantially constant temperature. For example, the first apparatus (first sub-module) 110A may include a temperature control unit, e.g., a heating element (such as a heat block, a heat plate, a heating coil, and/or the like), in contact with, or in the proximity of, the sample. In some variations, the processor 124 of the compute device 120 may be configured to control the current delivered to the temperature control unit (e.g., via a drive circuit) to establish and maintain the temperature of the heating element. Temperature modification (e.g., heating) of the sample may occur in the first and/or second apparatuses (sub-modules). For example, heating of samples in the second apparatus (second sub-module) 110B may be used to obtain denaturation/kinetic information about the sample such as fluorescence intensity and/or a derivative thereof.

In some variations, the systems for analyzing the stability of samples may generally include an optical module and a sample plate including one or more substrates. The one or more substrates may include a plurality of sample holders (e.g., cuvettes) and an inlet fluidly coupled to each of the plurality of sample holders. Additionally, the systems may include a clamp having an actuated (e.g., clamped) state in which it is configured to trap and pressurize air above the inlet. In these variations, the systems may comprise multiple modules and sub-modules configured to measure various sample parameters in a manner such that a single instrument may run several experiments sequentially or simultaneously.

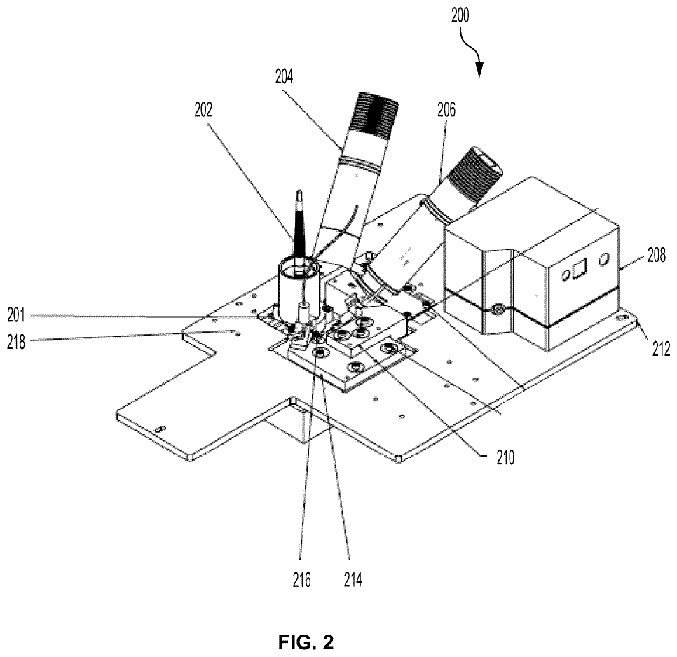

In one variation, the system may comprise an optical module that includes a first sub-module configured to measure fluorescence of a sample within at least a portion of the plurality of sample holders. The first sub-module may also measure static light scattering (SLS) of the sample within at least a portion of the plurality of sample holders, or the SLS may be measured using a separate, second sub-module. A third sub-module configured to measure dynamic light scattering (DLS) of a sample within at least a portion of the plurality of sample holders may further be included. In some instances, the systems may also include a temperature control unit having a heating element for heating the samples. For example, referring to FIG. 2, an exemplary optical module 200 for analyzing the stability of sample may include a fluorescence sub-module 202, which may be attached to other system components via a clamp 202, a blue (light) sub-module 204, a UV sub-module 206, and a DLS sub-module 210. The optical module 200 may also include a spectrometer 208, an optical plate 212, an optics camera 214, and a mirror 218. The systems may further include a display (not shown) configured to visually present data obtained from the optical module.

More specifically, the systems may be configured to measure one or more parameters indicative of stability including melting temperature (Tm), aggregation temperature (Tagg), isothermal stability, viral capsid stability, polydispersity, diameter, size distribution, sizing with thermal ramp, thermal recovery, viscosity, kp (diffusion interaction parameter), B₂₂ (second virial coefficient), G₂₂ (Kirkwood-Buff integral), and AG. For example, when biologics or gene vectors are being analyzed, their behavior over the entire fluorescence spectrum may be obtained. Here dyes may also be included and SLS performed at two wavelengths of light to monitor aggregation. In other instances, Tm and Tagg may be run at the same time to determine when unfolding may lead to aggregation. Furthermore, the system may take a DLS reading to obtain information relating to polydispersity, diameter, and/or size distribution of the substance of interest. In other variations, the systems may use SYBR Gold fluorescence to determine the temperature at which DNA or RNA may start to leak from viral vectors.

When proteins are to be analyzed in the samples, the systems may include an optical module containing a first sub-module for assessing protein stability using heat and detection of fluorescence. For example, the first module may be configured to measure the melting temperature (Tm) and the aggregation temperature (Tagg) to determine the stability of proteins under heat stress. This may be useful for comparing formulations and evaluating excipients that may stabilize proteins during thermal changes.

In addition to assessing proteins under heat stress, the first sub-module may also be configured to detect static light scattering (SLS), or system may include other sub-modules, e.g., a second sub-module, third sub-module, configured to detect SLS and/or dynamic light scattering (DLS). Using dynamic and/or static light scattering, the system may monitor protein size, polydispersity, and aggregation tendencies in response to temperature and/or environmental changes. This capability may enable assessment of factors such as colloidal stability and aggregation risk over extended experiments. Collectively, the modules or assemblies may support custom thermal profiles (isothermal, linear, stepped, sawtooth) and freeform experiments, allowing flexibility in the design of temperature ramps and hold times for in-depth analysis of protein behavior under specific conditions.

The system may collect and analyze data with analysis software, which may provide detailed profiles of protein unfolding, aggregation, and stability metrics. The software may also calculate parameters like Tm and Tagg, hydrodynamic diameter (e.g., via DLS), and second virial coefficients (e.g., B22), thus making the system a comprehensive stability assessment tool.

### SAMPLE PLATES

As mentioned above, intrinsic fluorescence, SLS, and DLS may be assessed by the systems described herein sequentially or simultaneously when assessing the stability of substances. In order to accomplish these assessments, it may be useful for a sample plate to include portions that are optically transparent to UV and blue light (e.g., with little to no autofluorescence due to excitation at 266 nm and 473 nm), non-interfering with the DLS acquisition (e.g., no flaws or autofluorescence from a 660 nm light source), and thermally stable at temperatures between about 15 degrees Celsius to about to about 120 degrees Celsius. Additionally, it may be useful for the sample plate to have good thermal transition to the samples that are contained within it.

The sample plates that may be used with the systems described herein may generally be configured to allow multiple experiments to be run on a single low volume sample with a single instrument. The low volume sample may have a volume between about 5.0 μl to about 10 μl, including all values and sub-ranges therein. For example, the volume may be about 5.0 μl, about 5.5 μl, about 6.0 μl, about 6.5 μl, about 7.0 μl, about 7.5 μl, about 8.0 ul, about 8.5 μl, about 9.0 μl, about 9.5 μl, or about 10 μl. In one variation, the volume of the sample may be about 9.0 ul. The low sample volume may help thermal conduction to be more efficient and responsive throughout the sample volume.

The sample plate may include a frame having length and a width coupled to the one or more substrates (chips). As used herein, the terms “substrate” and “chip” are used interchangeably throughout. Given that the sample plate may be used with fully or partially automated instruments and/or processes, they may be configured to include 96 sample holders (e.g., cuvettes), and be compatible with pipetting samples manually or with a standard automated fluid handler (e.g., the sample inlets are in the SBS standard positions and the frame in line with the standard for robotic plate grippers). As used herein, the terms “sample holder” and “cuvette” are used interchangeably throughout. The frame may have a length between about 120 mm and about 130 mm, including all values and sub-ranges therein. For example, the frame may have a length of about 120 mm, about 120.5 mm, about 121 mm, about 121.5 mm, about 122 mm, about 122.5 mm, about 123 mm, about 123.5 mm, about 124 mm, about 124.5 mm, about 125 mm, about 125.5 mm, about 126 mm, about 126.5 mm, about 127 mm, about 127.5 mm, about 128 mm, about 128.5 mm, about 129 mm, about 129.5 mm, or about 130 mm. In one variation, the length of the frame may be about 128 mm. The frame may have a width between about 80 mm and about 90 mm, including all values and sub-ranges therein. For example, frame may have a width of about 80 mm, about 80.5 mm, about 81 mm, about 81.5 mm, about 82 mm, about 82.5 mm, about 83 mm, about 83.5 mm, about 84 mm, about 84.5 mm, about 85 mm, about 85.5 mm, about 86 mm, about 86.5 mm, about 87 mm, about 87.5 mm, about 88 mm, about 88.5 mm, about 89 mm, about 89.5 mm, or about 90 mm. In one variation, the width of the frame may be about 85 mm. The frame may also have a thickness ranging from about 10 mm to about 15 mm, including all values and sub-ranges therein. For example, the frame may have a thickness of about 10 mm, about 10.5 mm, about 11 mm, about 11.5 mm, about 12 mm, about 12.5 mm, about 13 mm, about 13.5 mm, about 14 mm, about 14.5 mm, or about 15 mm. In some variations, the length of the frame may be about 128 mm, and the width of the frame may be about 85 mm.

Various types of polymers may be used to make the frame. It may be beneficial for the polymers employed to be non-fluorescent or have minimal fluorescence so as not to interfere with dynamic light scattering (DLS) measurements. In one variation, the frame may be made from Polycarbonate Acrylonitrile Butadiene Styrene (PC-ABS). It may also be beneficial for the color of the frame to be black in order to cut down on light reflection, but other colors with minimal light reflection may be used.

The sample plates may include any number of sample holders. For example, the sample plate may be sized and shaped to include 96 sample holders. However, in other variations, more or less sample holders may be included. When a sample of interest is to be tested, each sample holder may be loaded with a sample containing a target drug, protein, viral vector, or a combination thereof. The plurality of sample holders may be variously shaped. In some variations, the plurality of sample holders may have a diamond, rounded diamond, marquis, ovular, circular, rectangular, or square shape.

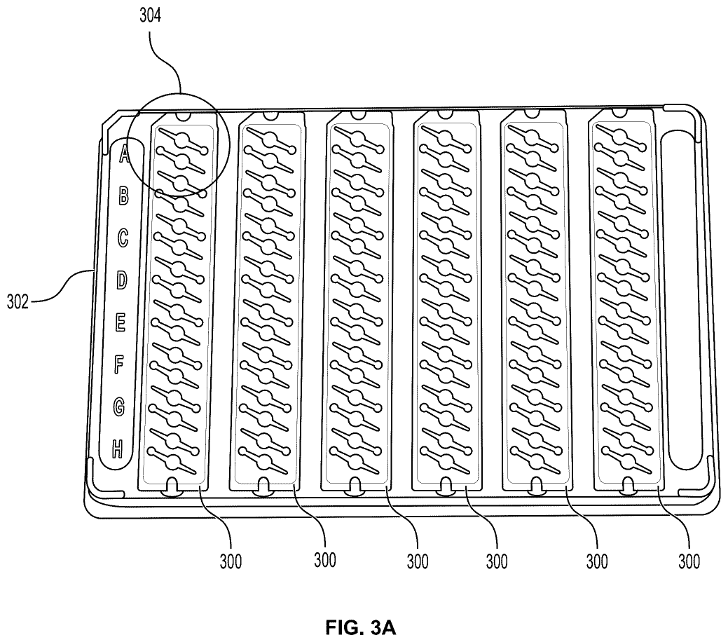

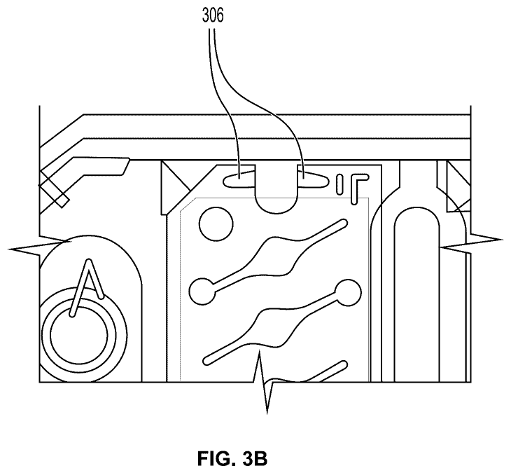

Some variations of the sample plate may include six chips attached to the frame. Attachment may be accomplished via one or more pins on the frame that fit into one or more notches in each of the substrates, and/or use of an adhesive. For example, as shown in FIG. 3A, six chips 300 may be fixedly attached to a frame 302. In FIG. 3B, which is an expanded view of the portion 304 of the sample plate circled in FIG. 3A, wing-shaped holes 306 (also circled in red and indicated by the red arrow) on either side of a pin of the frame may be provided to hold an adhesive used to attach the chips 300 to the frame 302. Any suitable adhesive may be employed. Use of the wing-shaped holes may be beneficial to achieving a strong adhesive seal while keeping the chips level. Although wing-shaped holes are illustrated in FIG. 3B, it is understood that the holes may be shaped differently.

When six chips are included in the sample plates, the chips may have a length between about 70 mm to about 80 mm, including all values and sub-ranges therein and a width between about 10 mm to about 15 mm, including all values and sub-ranges therein. For example, the chips may have a length of about 70 mm, about 71 mm, about 72 mm, about 73 mm, about 74 mm, about 75 mm, about 76 mm, about 77 mm, about 78 mm, about 79 mm, or about 80 mm. In one variation, the length of the chips may be about 79 mm. With respect to width, the chips may have a width of about 10 mm, about 10.5 mm, about 11 mm, about 11.5 mm, about 12 mm, about 12.5 mm, about 13 mm, about 13.5 mm, about 14 mm, about 14.5 mm, or about 15 mm. In one variation, the width of the chips may be about 13.5 mm. In some variations, the length of the chips may be about 79 mm, and the width of the chips may be about 13.5 mm. In these variations, each chip may include 16 cuvettes (and thus be able to hold 16 samples), and when six such chips are set on the frame, the sample plate may include 96 cuvettes/samples.

The one or more substrates may comprise a plurality of substrate layers. Any number of substrate layers may be included (e.g., two, three, four). The substrate layers may be fused using any suitable technique that does not interfere with optical readings and/or leech chemicals into the samples, e.g., when the samples are heated. For example, the substrate layers may be fused without the use of a bonding agent or film. Additionally, any material that does not interfere with fluorescence and optical readings, and which may form an atomically flat surface to optimize the transfer of heat to the samples may be used to make the substrate layers. For example, it may be useful for the optical read area of the substrates to be of sufficient optical quality to allow for static light scattering (SLS) and dynamic light scattering (DLS) detection with minimal interfering background scattering. Accordingly, use of a quartz material may be beneficial. In some variations, each of the plurality of substrate layers may be made of a quartz material. For example, JGS2 quartz or JGS1 quartz may be used. In other variations, polymer materials capable of effectively transmitting UV light, e.g., UV light between 280 nm and 660 nm and at a transmission rate of about 85% to about 95%, may be used. Exemplary polymers that transmit UV light include without limitation, cyclic olefin copolymer (COC), ultraviolet transmission (UVT) acrylic, and ultraviolet transmission (UVT) polycarbonate.

Each of the plurality of substrate layers may have a thickness between about 0.5 mm to about 1.0 mm, including all values and sub-ranges therein. For example, the thickness of each substrate layer may be about 0.5 mm, about 0.6 mm, about 0.7 mm, about 0.8 mm, about 0.9 mm, or about 1.0 mm. In one variation, each of the plurality of substrate layers may have a thickness of about 0.7 mm.

Each of the plurality of substate layers may be atomically flat, as mentioned above. For example, each substrate layer may have a total thickness variation (TTV) between 0 (zero) and about 5 μm (including all values and sub-ranges therein), a bow between 0 (zero) and about 15 μm (including all values and sub-ranges therein), and a warp between 0 (zero) and about 20 μm (including all values and sub-ranges therein). With respect to TTV, each substrate layer may have a TTV of 0 (zero), about 1.0 μm, about 1.5 μm, about 2.0 μm, about 2.5 μm, about 3.0 μm, about 3.5 μm, about 4.0 μm, about 4.5 μm, or about 5.0 μm. With respect to bow, each substrate layer may have a bow of about 0 (zero), 1.0 μm, about 2.0 μm, about 3.0 μm, about 4.0 μm, about 5.0 μm, about 6.0 μm, about 7.0 μm, about 8.0 μm, about 9.0 μm, about 10 μm, about 11 um, about 12 μm, about 13 μm, about 14 μm, or about 15 μm. With respect to warp, each substrate layer may have a warp of 0 (zero), about 1.0 μm, about 2.0 μm, about 3.0 μm, about 4.0 μm, about 5.0 μm, about 6.0 μm, about 7.0 μm, about 8.0 μm, about 9.0 μm, about 10 μm, about 11 μm, about 12 μm, about 13 μm, about 14 μm, about 15 μm, about 16 μm, about 17 μm, about 18 μm, about 19 μm, or about 20 μm. In some variations, each of the plurality of substrate layers may have a TTV less than about 5 μm, a bow of less than about 15 μm, and a warp of less than about 20 μm.

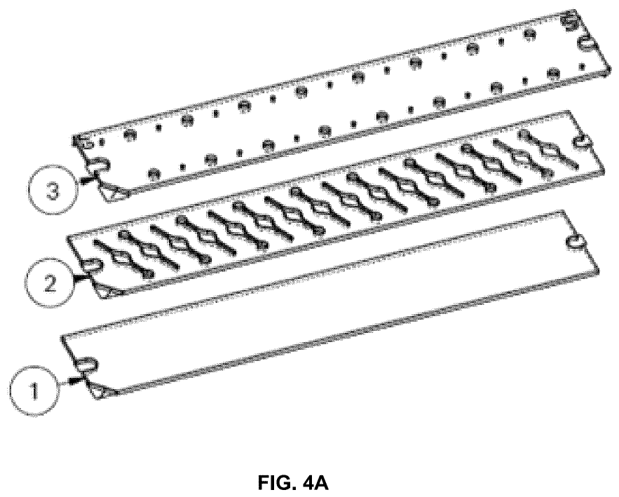

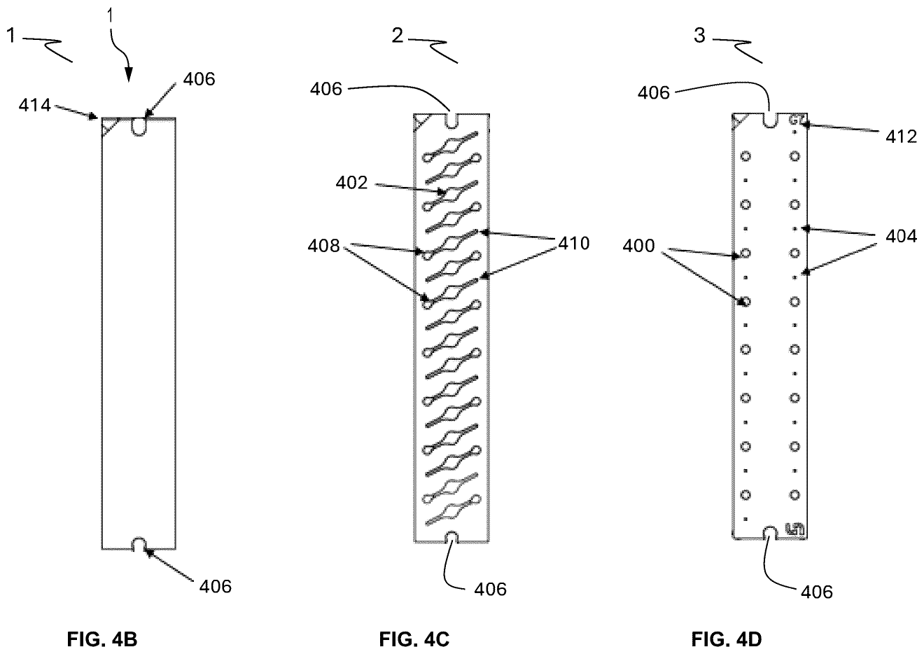

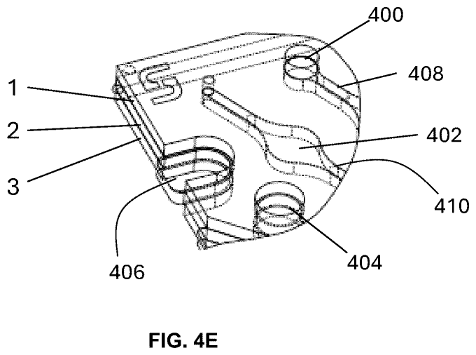

When the substrate includes three substrate layers, it may comprise a top layer 3, a base layer 1, and a middle layer 2 between the top and base layers, as shown in FIG. 4A. In this configuration, as shown in more detail in FIGS. 4B-4D, the top layer 3 may include sample loading features such as the inlet 400 for loading the sample on a first side of each sample holder (cuvette 402) and a vent outlet 404 on a second, opposite side of each cuvette 402, and the base layer 1 may comprise one or more notches 406 at an edge thereof configured to mate with one or more pins (not shown) on the sample plate (layers 2 and 3 may also include notches 406), and a tab 414 to identify a corner, e.g., a left corner of the chip. The middle layer 2 may include the plurality of cuvettes 402, and an inlet channel 408 and a vent channel 410 extending from each of the plurality of cuvettes 402. The inlet channel 408 may fluidly connect the inlet 400 to each of the plurality of cuvettes 402, and the vent channel 410 may fluidly connect a vent outlet 404 to each of the plurality of cuvettes 402, as shown in FIG. 4E. In some variations, each of the plurality of cuvettes and its corresponding inlet channel and vent channel may be sized and/or shaped to collectively hold a small sample volume, e.g., a sample volume of about 9.0 ul. A version number 412 may also be included on the top layer 3 of the chips.

Although the cuvettes shown in FIGS. 4A, 4C, and 4E are shaped like a diamond having rounded vertices, it is understood that other shapes may be used, e.g., circular, square, rectangular, rhombus, triangular, or ovular shapes. As described above, the volume of the cuvettes will generally be small, ranging from about 5 μl to about 10 μl, including all values and sub-ranges therein. In some variations, the volume of the cuvettes may be about 9.0 μl. The inlet channels and vent channels may have a width of about 0.5 mm and a length ranging from about 2.0 mm to about 4.0 mm, including all values and ranges therein. For example, the length of the inlet and vent channels may be about 2.0 mm, about 2.5 mm, about 3.0 mm, about 3.5 mm, or about 4.0 mm. The inlet and vent outlets may have a circular shape, or a different shape, if desired.

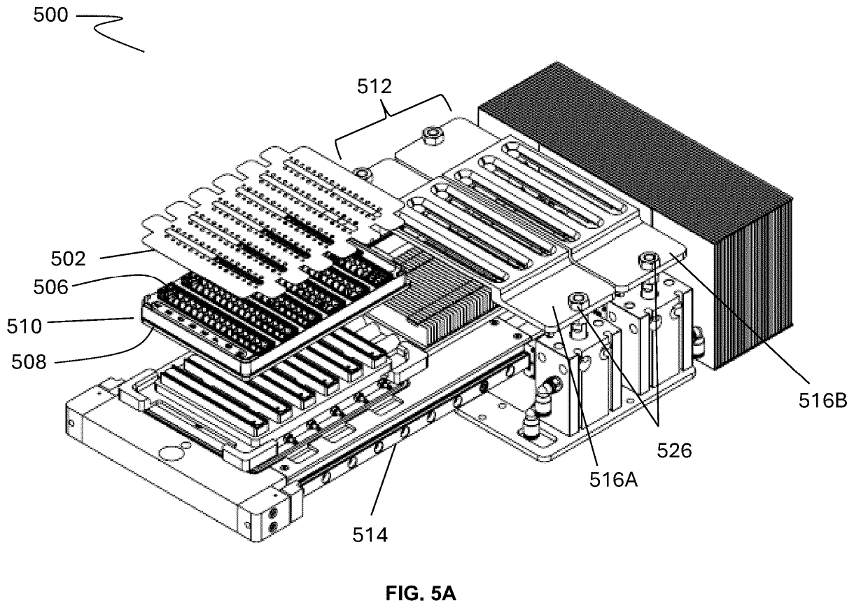

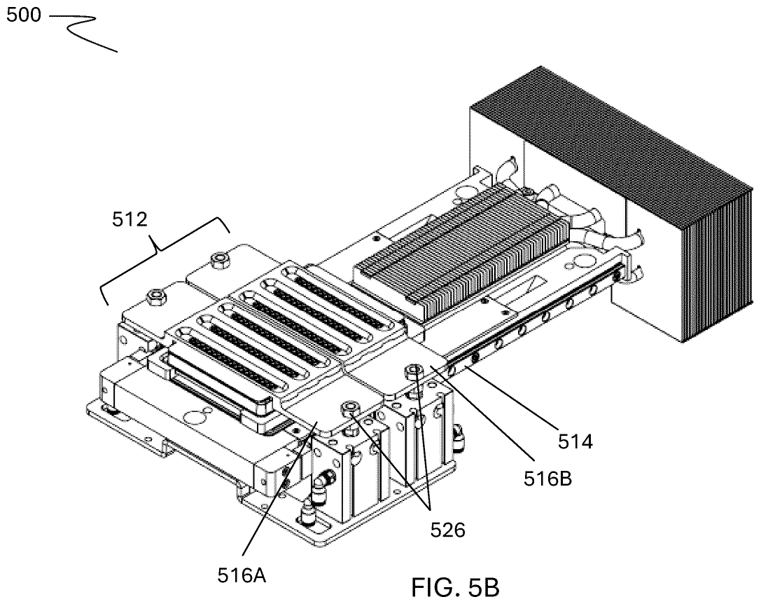

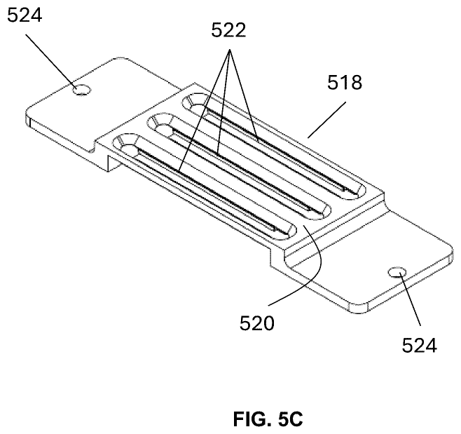

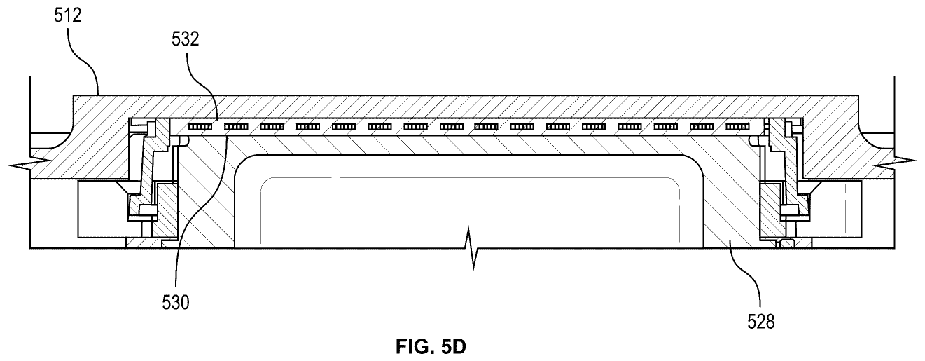

The samples being tested will typically require heating, e.g., heating to about 95 degrees Celsius. Accordingly, the systems and/or sample plates may generally include mechanisms to minimize evaporation and bubble formation. Bubble formation may cause data from the optical detection methods (e.g., fluorescence and light scattering) to be unusable. In addition, evaporate of the sample may change the concentration of the substance (e.g., the protein) and adversely affect results. In some variations, the systems may employ a plate seal configured to cover at least a portion of the one or more substrates and adhesively attach to a frame of the sample plate. For example, as shown in FIG. 5A, the plate seal 502 of system component 500 (e.g., optical module) may comprise one or more elongate openings 504 disposed over the plurality of sample holders 506 when the plate seal 502 is adhesively attached to the frame 508 of the sample plate 510.

In addition to the plate seal, the systems may include a clamp having an actuated state and a rest state. The clamp may comprise a plurality of pistons (e.g., pneumatic pistons) that allow the clamp in its actuated state to apply a force between about 300 kPa (3 bar) to about 500 kPa (5 bar), including all values and sub-ranges therein, to the sample plate to provide further sealing of the sample. For example, the clamp in its actuated state may apply a force of about 300 kPa, about 325 kPa, about 350 kPa, about 375 kPa, about 400 kPa, about 425 kPa, about 450 kPa, about 475 kPa, or about 500 kPa to the sample plate. At about 300 kPa (3 bar), the corresponding pressure on the sample plate is approximately 70 lbs.

In the actuated state, the clamp may trap and pressurize a column of air above an inlet fluidly coupled to each of the plurality of sample holders. The pressurized air may maintain a higher local pressure in the sample holders. In some variations, the clamp may also trap and pressurize air above a vent outlet fluidly coupled to each of the plurality of sample holders.

For example, referring to FIG. 5B, after the sample plate 510 is placed on stage 514 and moved to the read location, clamp 512 may transition from its rest state (as shown in FIG. 5A) to an actuated state (shown in FIG. 5B) over the sample plate 510 to cover the plate seal 502 and the plurality of samples in the sample plate 510. As shown in FIGS. 5A to 5C, the clamp 512 may include two clamp plates 516A and 516B, which may generally have the same structure. For example, as shown in FIG. 5C, a central portion 518 of each clamp plate may be raised to allow the sample plate to be placed beneath it. The top surface 520 of each clamp plate may also include elongated openings 522 that align with the elongated openings 504 of the plate seal 502 shown in FIG. 5A, and which allow interrogation of the samples and/or measurements to be taken from the samples. The clamp plates may also include openings 524 for coupling to pistons 526 (FIGS. 5A and 5B) (e.g., pneumatic pistons) that may move the clamp plates such that they contact the sample plate 510 and apply a force to the sample plate 510. Although four (4) pistons are shown in FIGS. 5A and 5B, any suitable number of pistons may be employed. Instead of compressed air, other mechanisms may also be used to move the pistons and transition the clamp from the rest state to the actuated state. In the actuated state, the clamp may apply a force between about 300 kPa (3 bar) to about 500 kPa (5 bar), including all values and sub-ranges therein, to the sample plate, as described above. The small amount of air trapped at the sample inlet and/or vent outlet by this additional sealing may provide a compressible layer capable of counteracting increases in pressure that occur when the samples are heated. For example, the trapped and pressurized air in contact with the samples may prevent the samples from outgassing or boiling during the thermal ramp.

The one or more substrates/substrate layers generally have an atomically flat surface to optimize the transfer of heat to the samples, as previously described. Sample plates having good thermal control may allow the temperature of the samples to be ramped in a controlled and predictable manner. Heating (and cooling) may be accomplished by any suitable temperature control system and/or device. In some variations, a Peltier heating and cooling system may be employed. For example, referring to the cross-sectional view of FIG. 5D, a copper block 528 may be used for transfer of heat to the sample plate 510 sitting directly on a top surface 530 thereof when clamp 512 in its actuated state applies a force, as described above, to the sample plate 510 and substrates 532.

### METHODS

In use, the samples may first be placed in any one of the sample plates described herein. An adhesive seal may then be placed over the sample plate to prevent evaporation. The adhesive seal may include a plurality of cutouts or openings Next, a clamp disposed above the sample plate applies a force to the top surface of the plate by pressing against it, such that the clamp further seals the samples within the sample holders of the plate to prevent evaporation during the thermal ramp. The sample plate may then be loaded into the instrument and aligned on a temperature-controlled stage. In some variations, 96 samples are loaded into the instrument. The Peltier heating and cooling component (temperature control unit) of the system may then enable precise temperature control from about 15 degrees Celsius to about 95 degrees Celsius (including all values and sub-ranges Attorney therein), with thermal ramps of 0.1 to 10 degrees Celsius per minute. For measurements, the system in some variations may also use a 280 nm UV laser for fluorescence excitation, a 470 nm laser for SLS, and a 660 nm laser for DLS. It is understood that other lasers may be used for fluorescence, SLS, and DLS.

Other methods for analyzing the stability of samples are also described herein. These methods may generally include loading a sample into a a plurality of sample holders of a sample plate, where the sample plate includes a frame and one or more substrates coupled to the frame, and where the one or more substrates includes a top layer, a base layer, and a middle layer between the top and base layers. A clamp may then be actuated to apply a force to the sample plate and form a seal that pressurizing air trapped above an inlet of the each of the plurality of sample holders. The force applied by the clamp may be between about 300 kPa (3 bar) to about 500 kPa (5 bar), including all values and sub-ranges therein. In some instances, the method may include pressurizing air trapped above a vent outlet of each of each of the plurality of sample holders. The loading of the samples may be partially automated or fully automated. The samples of interest may include any suitable target substance to be assessed, including drug, proteins, viral vectors, biologics, chemicals, or combinations thereof.

Some variations of the method may further include adhesively attaching a plate seal on the sample plate to cover at least a portion of the one or more substrates, e.g., when the method includes heating of the samples. Additionally, the methods may include one or more steps of detecting fluorescence of the sample, detecting static light scattering (SLS), and detecting dynamic light scattering (DLS) of the sample. The data obtained from these various detection methods may be displayed on a screen as, e.g., numerical data, lists, charts, graphs, or other visual (e.g., color-coded) representations of the information.

In other variations, the method may include receiving a sample, the sample including a drug (e.g., a protein) component. The method may include detecting a thermodynamic state of the sample. In some variations, a denaturing agent may be added to the sample prior to detecting the thermodynamic state. The method may also include modifying the temperature of the sample and detecting, in response to the modifying the temperature, a rate of denaturation of a protein in the sample. The method also includes computing the thermodynamic information and the kinetic information using one or more processors, to provide an indication of stability of the protein component of the sample. As mentioned above, instead of proteins, the one or more processors may also base its analysis of stability on information obtained for other substances such as viral vectors, biologics, or chemicals.

The foregoing description, for purposes of explanation, used specific nomenclature to provide a thorough understanding of the invention. However, it will be apparent to one skilled in the art that specific details are not required in order to practice the invention. Thus, the foregoing descriptions of specific embodiments of the invention are presented for purposes of illustration and description. They are not intended to be exhaustive or to limit the invention to the precise forms disclosed; obviously, many modifications and variations are possible in view of the above teachings. The embodiments were chosen and described in order to explain the principles of the invention and its practical applications, they thereby enable others skilled in the art to utilize the invention and various embodiments with various modifications as are suited to the particular use contemplated.

1. A system for analyzing the stability of samples comprising:
an optical module;
a sample plate comprising one or more substrates, the one or more substrates comprising a plurality of sample holders and an inlet fluidly coupled to each of the plurality of sample holders; and
a clamp having an actuated state, wherein in the actuated state the clamp is configured to trap and pressurize air above the inlet.

2. The system of claim 1, wherein the optical module comprises a first sub-module configured to measure fluorescence of a sample within at least a portion of the plurality of sample holders.

3. The system of claim 1, wherein the optical module comprises a second sub-module configured to measure static light scattering (SLS) of a sample within at least a portion of the plurality of sample holders.

4. The system of claim 1, wherein the optical module comprises a third sub-module configured to measure dynamic light scattering (DLS) of a sample within at least a portion of the plurality of sample holders.

5. The system of claim 1, further comprising a temperature control unit.

6. The system of claim 1, wherein the sample plate further comprises a frame having length and a width coupled to the one or more substrates.

7.-13. (canceled)

14. The system of claim 1, wherein the one or more substrates comprises a plurality of substrate layers.

15.-18. (canceled)

19. The system of claim 14, wherein each of the plurality of substrate layers has a thickness between about 0.5 mm to about 1.0 mm.

20. (canceled)

21. The system of claim 14, wherein the plurality of substrate layers includes a top layer, a base layer, and a middle layer between the top and base layers.

22. The system of claim 21, wherein the top layer comprises the inlet and a vent outlet.

23. (canceled)

24. The system of claim 21, wherein the middle layer comprises the plurality of sample holders and an inlet channel and a vent channel extending from each of the plurality of sample holders.

25. The system of claim 24, wherein the inlet channel fluidly connects the inlet to each of the plurality of sample holders.

26. The system of claim 24, wherein the vent channel fluidly connects a vent outlet to each of the plurality of sample holders.

27. (canceled)

28. The system of claim 1, wherein the clamp comprises a plurality of pneumatically controlled pistons.

29. The system of claim 1, wherein the clamp in its actuated state applies a force of about 300 kPa (3 bar) to about 500 kPa (5 bar) to the sample plate.

30. The system of claim 1, wherein in the actuated state the clamp is further configured to trap and pressurize air above a vent outlet fluidly coupled to each of the plurality of sample holders.

31.-32. (canceled)

33. The system of claim 1, further comprising a display configured to visually present data obtained from the optical module.

34. The system of claim 1, further comprising a sample within the plurality of sample holders, wherein the sample comprises a drug, a protein, a viral vector, or a combination thereof.

35.-36. (canceled)

37. A sample plate comprising:
a frame; and
a plurality of substrates coupled to the frame,
wherein each of the plurality of substrates comprises a top layer, a base layer, and a middle layer between the top and base layers, and
the middle layer comprises a plurality of sample holders, and the top layer comprises an inlet and a vent outlet fluidly connected to each of the plurality of sample holders.

38.-43. (canceled)

44. A method for analyzing the stability of samples comprising:
loading a sample into a plurality of sample holders of a sample plate, wherein the sample plate comprises a frame and one or more substrates coupled to the frame, and wherein the one or more substrates comprises a top layer, a base layer, and a middle layer between the top and base layers;
actuating a clamp to apply a force to the sample plate and form a seal; and
pressurizing air trapped above an inlet of the each of the plurality of sample holders.

45.-53. (canceled)
